(19) United States
(12) Patent Application Publication
(10) Pub. No.: US 2009/0036880 A1
(43) Pub. Date: Feb. 5, 2009

(54) Device and Method for Changing a Lens Implanted Into an Eye

(76) Inventors: Mark Bischoff, Bad Berka (DE); Michael Kempe, Jena (DE); Markus Strehle, Jena (DE); Walter Wrobel, Jena (DE)

Correspondence Address: PATTERSON, THUENTE, SKAAR & CHRISTENSEN, P.A., 4800 IDS CENTER, 80 SOUTH 8TH STREET, MINNEAPOLIS, MN, 55402-2100, US

(21) Appl. No.: 11/988,399

(22) PCT Filed: Jul. 5, 2006
(86) PCT No.: PCT/EP2006/006564

§ 371 Date: Jan. 7, 2008

(30) Foreign Application Priority Data
Jul. 8, 2005 (DE) 10 2005 032 041.4

(51) Int. Cl. A61F 9/011

(52) U.S. Cl. 606/13

(57) ABSTRACT

The invention relates to a device for altering an optical and/or mechanical property of a lens that is implanted in an eye, the device including a laser device, which has a laser beam source that provides a pulsed laser beam and an optical unit, which impinges on the implanted lens with the pulsed laser beam. The device also includes a control device, which controls the laser device such that the optical and/or mechanical property of the lens is altered on the basis of non-linear interaction between the laser beam and the lens material.

The invention relates to a device and a method for changing an optical and/or mechanical property of a lens implanted into an eye.

Such a method is described, for example, in WO 00/41650 A1, in which method the lens to be implanted has a special design. Said lens comprises a first polymer matrix in which a compound modulating the refractive index is dispersed, wherein polymerization can be effected by means of UV radiation. Therefore, according to this method, UV radiation is applied to the lens implanted into the eye (intraocular lens) so as to effect the desired change in refractive index. Although this method is contactless, it has the disadvantage that the UV radiation passes through the cornea during treatment and may, thus, damage the cornea. In particular, this method requires carrying out an irradiation in any case, even if no correction is required, because in this case, fixing of the existing optical properties of the implanted lens is necessary.

In view thereof, it is an object of the invention to provide a device and a method for changing an optical and/or mechanical property of a lens implanted into an eye, wherein the change can be effected in a contactless manner and any damage to the cornea can be avoided.

According to the invention, this object is achieved by a device for changing an optical and/or mechanical property of a lens implanted into an eye, said device comprising a laser device which includes a laser radiation source providing pulsed laser radiation and an optical unit applying said pulsed laser radiation to the implanted lens, as well as a control device controlling the laser device such that a lasting change of the optical and/or mechanical lens property is effected on the basis of a non-linear interaction between the laser radiation and the material of the lens. The non-linear interaction between the laser radiation and the material of the lens allows the use of laser radiation having a wavelength which does not harm the cornea. Preferably, laser radiation in the near-infrared spectral region (greater than 750 nm) is used. The cornea and also the intraocular lens are transparent for this wavelength as long as only linear effects are taken into consideration. However, two- or multiple-photon absorptions may occur which will then cause the desired change of the lens property.

In order to provide the pulsed-laser radiation intensity required for the non-linear interaction, it is preferred that the laser radiation source provide the laser pulses with a pulse duration of less than 1 ps or less than 500 fs, in particular less than 100 fs.

In a preferred embodiment, the control device controls the laser device such that there is a non-linear interaction, but no optical breakthroughs. This is preferably effected by controlling the radiation intensity, because as the intensity increases, multi-photon absorptions occur first, and then, if the power density of the radiation exceeds a threshold, an optical breakthrough occurs at which a plasma bubble is produced in the material. Said plasma bubble grows due to expanding gases after forming the optical breakthrough. If the optical breakthrough is not maintained, the gas generated in the plasma bubble will be absorbed by the surrounding material and will disappear again. If a plasma is generated at a material interface which may even be located within a material structure, material removal is effected from said interface. This is then referred to as photoablation. In connection with a plasma bubble separating previously connected material layers, one usually speaks of photodisruption. For the sake of simplicity, all such processes are summarized here by the term optical breakthrough, i.e. this term includes not only the actual optical breakthrough, but also the effects resulting therefrom in the material.

Now, if the laser device is controlled such that no optical breakthroughs appear, an extremely accurate and slight change in the lens properties is possible.

In particular, the imaging optics comprise a deflecting unit by which the laser radiation can be focused in the lens and this focal point (spot) can be moved within the lens. By suitable local changes in the lens properties, the desired macroscopic modification of the lens property can be effected (for example, alteration of the refractive index, of the lens shape and/or of the elasticity of the lens). Spot sizes of 30 μm are possible, and the depth resolution may also be approximately 30 μm. For shifting in the first spatial direction (usually the z direction), the deflecting unit may preferably comprise a zoom lens which is provided as an adjustable telescope, and for the other two spatial directions (usually the x and y directions), it may comprise two oscillating mirrors with crossed axes of rotation. Thus, the intraocular lens can be altered or structured, respectively, in three dimensions to set the desired lens property.

The intensity required to cause the non-linear interaction which is not yet an optical breakthrough can be 10 to 100 times lower than the intensity required to produce optical breakthroughs. If a laser device is used by which optical breakthroughs are normally generated, the lower required intensity may be used such that the laser radiation is deflected or scanned at a higher speed so that the treatment duration can be considerably reduced or that focusing is less strong or that several foci are generated at the same time.

Of course, it is also possible to control the laser device using the control device such that optical breakthroughs occur. In this case, the optical breakthroughs are preferably generated such that one or more bubble layers form. This is particularly preferred in the case of liquid-filled or gel-filled intraocular lenses, where the lens material is gas-permeable, but impermeable for the liquid or the gel, respectively, of the intraocular lens.

Further, the optical unit may comprise imaging optics by means of which the laser radiation is spatially modulated and then imaged onto the implanted lens. In this case, the change in the lens property can be effected especially quickly. However, it should be noted that the required photon density for the non-linear interaction must not cause any harm to the eye. In order to reduce the photon density, imaging may be effected such that the implanted lens is not irradiated in its entirety, but parts of the implanted lens are respectively irradiated after one another and thus changed.

The object is further achieved by a method for changing an optical and/or mechanical property of a lens implanted into an eye, said method comprising the steps of:

measuring the deviation of at least one optical property of the implanted lens from a predetermined value,

determining the required change of an optical and/or mechanical property of the implanted lens in order to reduce the measured deviation, and

applying pulsed laser radiation to the implanted lens such that the required change of the optical and/or mechanical lens property is caused by the non-linear interaction between the laser radiation and the material of the lens. Said non-linear interaction allows the use of laser radiation having a wavelength which is transmitted by the cornea and accordingly does not harm the cornea.

In particular, laser radiation having a wavelength in the near-infrared range, i. e. of greater than 750 nm, is used.

The pulse duration of the laser radiation can be less than 1 ps, further less than 500 fs, in particular less than 100 fs. The use of such pulses allows to achieve the required intensity for the non-linear interaction.

Irradiation can be effected such that, although a non-linear interaction occurs, there will be no optical breakthroughs. In this case, an extremely precise local change of a material property of the implanted lens is possible, allowing to realize the desired macroscopic change of the lens property.

Of course, the method may also be carried out such that optical breakthroughs appear. In this case, the desired change of the lens property is achieved by the material removal occurring in the case of optical breakthroughs, with the resulting gas diffusing outward in the intraocular lens. For liquid-filled or gel-filled lenses, an outer lens material is used which is gas-permeable, but not permeable for the enclosed liquid or gel. Materials which can be used for such lenses include, for example: CAB (cellulose acetobutyrate), polycon (a copolymer of 35% silicone and PMMA, pentamethyldisiloxanyl methylmethacrylate+methylmethacrylate copolymerisate), menicon (synthesized copolymerisate of polyols and methacrylmethylsiloxane), conflex (polymeric alloy of CAB and copolymeric EVA−ethylvinyl acetate), a mixture of silicone and vinyl pryrrolidol, HEMA (2-hydroxyethylmethacrylate), hydroxypropylmethacrylate, HEMA hydrogels (cross-linked homopolymer of hydroxymethacrylate comprising 38-42% water) and silicone (polysiloxanes). The optical breakthroughs can be produced such that one or more layers of gas bubbles are generated which diffuse outward and, thus, disappear from the implanted lens, thereby causing a change in the shape of the implanted lens.

Preferably, the laser radiation is focused into the implanted lens, and then the focus is moved within the lens. This movement can be effected in three dimensions, so that three-dimensional structuring or changing of the lens property can be carried out.

Further, it is possible to spatially modulate the laser radiation and then image it onto the implanted lens such that the change of the lens property can be carried out quickly. In doing so, either the entire lens can be irradiated at once, or several parts of the lens are irradiated after one another.

The invention will be explained in more detail below, by way of example and with reference to the drawings, wherein:

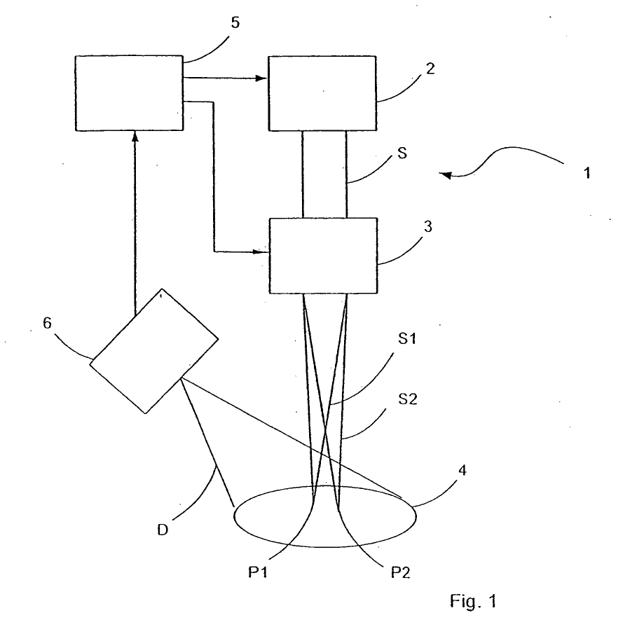

FIG. 1 shows a schematic view of a first embodiment of the device according to the invention, and

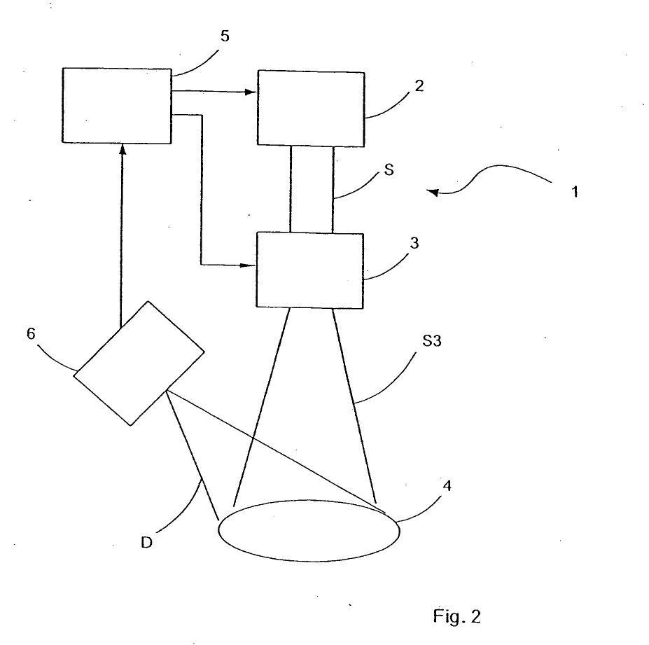

FIG. 2 shows a schematic representation of a second embodiment of the device according to the invention.

The device for changing an optical and/or mechanical property of a lens implanted into an eye comprises a laser device 1 containing a laser radiation source 2. In this case, the laser radiation source 2 is a TiSa laser, which emits laser pulses S having a wavelength of 780 nm and a pulse duration of 10 fs. The pulse shape and, in particular, the pulse duration can be set by spatially splitting the spectral components of a generated pulse and then providing different optical path lengths for the spatially split spectral components of the pulse and subsequently combining the spectral components in space. Such a procedure is described, for example, in T. Baumert et al., Applied Physics B 65, pages 779-782, 1997, “Femtosecond pulse shaping by an evolutionary algorithm with feedback” and in T. Brixner et al., Applied Physics B70 [Suppl.], pages 119-124, 2000, “Feedback-controlled femtosecond pulse shaping”. The contents of both publications are herewith incorporated by reference in the present application.

Further, the laser device 1 contains an optical unit 3, which is arranged following the laser radiation source 2 and which focuses (S1, S2) the laser radiation S from the laser radiation source 2 and can deflect said radiation in three spatial directions. The schematic view of FIG. 1 shows two different focus positions P1 and P2 within an intraocular lens 4. The intraocular lens is already implanted into the eye (not shown).

The device further comprises a control device 5, which controls the laser device 1 such that a non-linear optical interaction occurs at the focal points P1, P2. Now, the laser device 1 is controlled such that, due to the non-linear interaction at the points P1 and P2, the desired change of the optical and/or mechanical lens property occurs. The optical lens property may be, for example, the refractive index of the lens. The mechanical property of the lens may be, for example, its shape and/or its rigidity or elasticity. The lens may consist of one single material or of several materials. In particular, the lens may contain a material showing a structural change and/or a change in cross-linking due to the non-linear interaction.

Particularly suitable lens materials are such materials whose absorption edge on the short-wavelength side of the visible spectrum (i. e. the UV absorption edge) is at approximately the 1/n^{th} wavelength of the laser radiation used. In many cases, such materials have a relatively large effective cross-section of n-photon absorption. Of course, the corresponding wavelength of the laser radiation may also be selected in the near-infrared range, depending on the UV absorption edge of the lens material used, such that it is n times the wavelength of the UV absorption edge (with n being an integer greater than 1).

The interaction may be effected such that no optical breakthroughs occur yet. In this case, a very precise change of the lens property is possible. As an alternative, it is possible to select the intensity of the laser radiation such that optical breakthroughs do occur.

The device preferably also comprises a measuring device 6 by which the imaging properties of the implanted lens 4 can be measured, as schematically indicated by the cone beam D. After carrying out the measurement of the imaging properties, the desired correction or change, respectively, is then calculated (e. g. by the control device) and is then carried out by means of the device for changing an optical and/or mechanical property of the intraocular lens.

FIG. 2 shows another embodiment of the device. This embodiment differs from the device of FIG. 1 in that no laser beam is deflected, thus moving a focal point within the intraocular lens 4, but a spatially modulated laser beam S3 is imaged onto the lens 4 by means of the optical unit 3 so that the change of the optical and/or mechanical property of the intraocular lens 4 is carried out at once.

In order to carry out the method for changing an optical and/or mechanical property of a lens implanted into an eye, the intraocular lens 4 implanted into the eye is measured first, according to one embodiment, so as to detect patient-specific aberrations caused, for example, by individual deviations of the cornea from its ideal shape or by positioning errors of the implanted lens. Thus, this measurement allows to determine the deviation of at least one optical property of the implanted lens from a predetermined desired value. Depending on the deviation, the required change of an optical and/or mechanical property of the intraocular lens 4 is then determined. This determining step may be carried out, for example, by the measuring device 6, the control device 5 or another computer not shown. The data are then provided to the control unit 5, unless the control unit 5 has effected said determination itself, which then controls the laser device 1 such that the desired non-linear optical interaction between the pulsed laser radiation and the material of the intraocular lens occurs. Since the laser radiation used is in the infrared range, damage to the cornea as well as to the rest of the eye can be safely avoided.

Of course, it is also possible to perform the above-described steps (namely measuring step, determining step, irradiating step) several times in succession in order to achieve an optimum correction, if possible.

Further, prior to the first measuring step, the method may also include the step of implanting the lens into the eye.

1-17. (canceled)

18. A device for changing an optical and/or mechanical property of a lens implanted into an eye, said device comprising
a laser device including a laser radiation source providing pulsed laser radiation and an optical unit applying said pulsed laser radiation to the implanted lens, and
a control device, which controls the laser device such that a change of the optical lens properties, mechanical lens property or both of the foregoing is effected due to a non-linear interaction between the laser radiation and the material of the lens.

19. The device as claimed in claim 18, wherein the laser radiation source provides the laser radiation with a wavelength of greater than about 750 nm.

20. The device as claimed in claim 18, wherein the laser radiation source provides the laser radiation with a pulse duration of less than about 500 femtoseconds

21. The device as claimed in claim 18, wherein the laser radiation source provides the laser radiation with a pulse duration of less than less than about 100 femtoseconds.

22. The device as claimed in claim 18, wherein the control device controls the laser device such that optical breakthroughs occur in the lens material.

23. The device as claimed in claim 18, wherein the control device controls the laser device such that gas bubbles are produced in the lens material, which diffuse outwardly, thus causing a change in the shape of the implanted lens.

24. The device as claimed in claim 18, wherein the control device controls the laser device such that a non-linear interaction occurs, but no optical breakthroughs appear.

25. The device as claimed in claim 18, wherein the optical unit comprises imaging optics by which the laser radiation is imaged onto the implanted lens.

26. The device as claimed in claim 18, wherein the optical unit comprises a deflecting unit by which the laser radiation is focused into the lens and is moved therein.

27. A method for changing an optical and/or mechanical property of a lens implanted into an eye, said method comprising the steps of:
measuring the deviation of at least one optical property of the implanted lens from a predetermined value;
determining the required change of an optical property, a mechanical property or a combination of the foregoing of the implanted lens in order to reduce the measured deviation,
applying pulsed laser radiation to the implanted lens, said irradiation being applied such that the required change of the optical and/or mechanical lens property is caused by the non-linear interaction between the laser radiation and the material of the lens.

28. The method as claimed in claim 27, wherein laser radiation is applied at a wavelength of greater than about 750 nm.

29. The method as claimed in claim 27, wherein laser radiation is applied at a pulse duration of less than about 500 femtoseconds.

30. The method as claimed in claim 27, wherein laser radiation is applied at a pulse duration of less than about 100 femtoseconds.

31. The method as claimed in claim 27, wherein irradiation is effected such that optical breakthroughs appear in the lens material.

32. The method as claimed in claim 27 wherein irradiation is effected such that gas bubbles are produced in the implanted lens, which diffuse outwardly, thus causing a change in the shape of the implanted lens.

33. The method as claimed in claim 27, wherein irradiation is effected such that a non-linear interaction occurs, but no optical breakthroughs appear in the lens material.

34. The method as claimed in claim 27, wherein the laser radiation is spatially modulated and is then imaged onto the implanted lens.

35. The method as claimed in claim 27, wherein the laser radiation is focused into the implanted lens, and the focus is moved within the implanted lens.

36. The method as claimed in claim 27, wherein the lens is implanted into the eye prior to the measuring step.
